(19) United States
(12) Patent Application Publication
(10) Pub. No.: US 2003/0004142 A1
(43) Pub. Date: Jan. 2, 2003

(54) Use of NSAIDs for prevention and treatment of cellular abnormalities of the lung or bronchial pathway

(76) Inventors: Christopher P. Prior, Rosemont, PA (US); Drore Eisen, Cincinnati, OH (US); Louis Herlands, Cambridge, MA (US)

Correspondence Address: CHERYL H AGRIS PHD, PO BOX 806, PELHAM, NY, 10803, US

(21) Appl. No.: 10/124,893

(22) Filed: Apr. 17, 2002

Related U.S. Application Data
(60) Provisional application No. 60/284,731, filed on Apr. 18, 2001.

(51) Int. Cl. A61K 31/60; A61K 31/415; A61K 31/405; A61K 31/192

(52) U.S. Cl. 514/165; 514/406; 514/420; 514/570

(57) ABSTRACT

The invention is directed to uses of non-steroidal anti-inflammatory drugs (NSAIDs) for the treatment and prevention of cellular abnormalities of the lung or bronchial pathway.

This application claims priority from U.S. provisional application serial No. 60/284,731, filed Apr. 18, 2001, the contents of which are incorporate herein by reference.

### 1. FIELD OF THE INVENTION

The invention is directed to uses of non-steroidal anti-inflammatory drugs (NSAIDs) for the treatment and prevention of cellular abnormalities of the lung or bronchial pathway, wherein said NSAIDs are selected from the group consisting of COX-1 selective cyclooxygenase inhibitors, nonselective cyclooxygenase inhibitors, and partially selective cyclooxygenase-2 (COX-2) inhibitors.

### 2. BACKGROUND OF THE INVENTION

2.1. Pulmonary Related Diseases

In the United States, there has been a steady rise in the age-adjusted national death rate from pulmonary related diseases. The overwhelmingly predominant contributor to this trend is lung cancer. Currently about 8% of all deaths in the industrialized world are attributed to lung cancer. An estimated 155,000 new cases of lung cancer are currently diagnosed each year in the United States, and about 142,000 will die of the disease, about 1 death every 4 minutes making lung cancer is the most common form of cancer death. Only about 10% of the patients currently diagnosed with lung cancer will survive beyond 5 years. The survival rate has not improved substantially over the past 20 years.

Approximately 85% of all lung cancers are related to smoking and in long-term heavy smokers an elevated risk of developing lung cancer persists after smoking cessation. In the United States, there are approximately 45 million current smokers and an equal number of former smokers at risk of lung cancer.

Lung cancer, like other epithelial cancers, is preceded by a series of precursor lesions. It has been shown that lung cancer develops through a series of progressive stages from mild, moderate to severe atypia (intraepithelial neoplasia and dysplasia), carcinoma in situ (CIS), and then invasive cancer. Drugs for preventing cancer by treating pre-invasive precursor lesions are sorely needed.

Lung cancer, or bronchial carcinoma, refers strictly to tumors arising from the major airways (bronchi) and pulmonary parenchyma (bronchioles, alveoli, and supporting tissue), as opposed to those metastasizing from other sites. The four major forms of lung cancer, squamous cell carcinoma (SCC), adenocarcinoma (AC), large cell anaplastic carcinoma (LCAC), and small cell anaplastic carcinoma (SCAC), account for 98% of pulmonary malignancies. Although lung cancer can occur anywhere in the lungs, about three-quarters of primary lung cancers occur in and/or on the bronchial walls within the first three bronchial generations, i.e., near or proximal to the hilus, the region where the airways and major vessels enter and leave each lung. A smaller percentage occurs in more distal areas of the parenchyma. Many tumors occur near the carina, at the junction of the right and left bronchi with the trachea, presumably due to increased deposition of inhaled carcinogens. Squamous cell carcinoma tumors, the most common histological type, making up 30-40% of lung tumors, arise inside the surface layer of the bronchial wall and then invade the wall and adjacent structures. Squamous cell carcinomas tend to be relatively localized with fewer tendencies than the other lung cancer tumors to metastasize. Furthermore, squamous cell carcinoma has a precancerous stage where abnormal cells may be detected in the sputum. Adenocarcinoma tumors, also comprising 30-40% of lung cancers, occur in the mid- to outer third of the lung in about three-quarters of the cases. Adenocarcinomas tend to metastasize widely and frequently to other lung sites, the liver, bone, kidney, and brain- Small cell cancer, accounting for about 20% of all lung cancer, is the most aggressively metastatic and rapidly growing, and can begin near the hilus or in the lung periphery. Large cell tumors account for only a few percent of lung cancer and can occur anywhere in the lung. “Local failure,” where primary tumors spread to mediastinal lymph nodes, pleura, adrenal glands, bone, and brain, is common with adenocarcinoma, small cell anaplastic carcinoma, and large cell anaplastic carcinoma, and less so in squamous cell carcinoma.

The current “curative” treatment for lung cancer is surgery, but the option for such a cure is given to very few. Only about 20% of lung cancer is resectable, and out of this number less than half will survive five years. However, the survival rate is low when cancer recurs. Generally, chemotherapy is used to treat such recurring cancers. Radiation therapy (RT) is the standard treatment for inoperable non-small cell cancer, and chemotherapy (alone or with radiation therapy) is the treatment of choice for small cell and other lung cancer with wide metastasis. Recent advances in treatment have increased the life expectancy of patients with small cell lung cancer from 2 months to about 2 years. Patients with clinically localized but technically unresectable tumors represent a major problem for the radiotherapist, accounting for an estimated 40% of all lung cancer cases.

Adjunctive hyperthermia, the use of deep heating modalities to treat tumors, is being used increasingly to augment the therapeutic efficacy of radiotherapy and chemotherapy in cancer treatment. It has been estimated that eventually hyperthermia will be indispensable for 20 to 25% of all cancer patients. Hyperthermia clinical research is increasingly showing the importance of using specialized heating equipment to treat specific anatomical locations and sites rather than devices with more general-purpose heating capabilities. Unfortunately, current hyperthermia devices are ill suited to

providing deep, localized heating of lung cancer. Because of this limitation, very few applications of localized lung hyperthermia have been recorded in the literature.

2.2. NSAIDs

Though NSAIDs have been classically categorized according to their chemical structure, since the discovery of COX-2, they are increasing being categorized according to their COX-2 selectivity. Categories of NSAIDs are usually referred to COX-1 selective, nonselective, COX-2 preferential and COX-2 selective. COX-1 selective NSAIDs include but are not limited to flurbiprofen, ketoprofen, fenoprofen, piroxicam and sulindac. Nonselective inhibitors include but are not limited to aspirin, ibuprofen, indomethacin, ketorolac, naprosen, oxaprosin, tenoxicam and tolmetin. Relatively COX-2 selective inhibitors include but are not limited to diclofenac, etodolac, meloxicam, nabumetone, nimesulide and 6-MNA. Highly selective COX-2 inhibitors include celecoxib, rolfecoxib and other drugs like L-743337, NS-398 and SC 58125.

Experimental work has shown that increased amounts of prostaglandins and COX-2, a key enzyme involved in the prostaglandin synthetic pathway, are commonly found in a wide range of premalignant tissues and malignant tumors including cervical dysplasia and cancer.

Extensive evidence from genetic and pharmacological studies indicate that COX-2 is mechanistically linked to the development of cancer (see, for example, Dannenberg et al., The LANCET Oncology 2:544-551 (2002).

Elevated prostaglandin and COX-2 levels substantially contribute to carcinogenesis by inhibiting apoptosis (see Tsujii and DuBois Cell 83, 493-501 (1995)) and stimulating angiogenesis (see Tsujii et al., Cell 83, 493-501 (1995); Williams et al., J. Clin Invest 105, 1589-94 (2000) and Masferrer et al., Cancer Research 60, 1306-11 (2000).

Although there is substantial evidence that overexpression of COX-2 is linked to tumorigenesis, it is not clear whether the antitumor effects of NSAIDs entirely result from the inhibition of COX-2 activity. COX-independent mechanisms as ell as the inhibition of COX-1 may contribute to the antitumor effect of NSAIDs. COX-independent mechanisms of NSAIDs have been the subject of a recent workshop (see Hwang et al. Neoplasia 2, 91-97 (2002).

Highly selective cyclooygenase-2 inhibitors have been developed to minimize gastric side effects that can occur when non-selective COX inhibitors are administered peroral. However, there is no data that demonstrates their superiority over selective cyclooxygenase-1 inhibitors, nonselective cyclooxygenase inhibitors, and partially selective cyclooxygenase-2 (COX-2) inhibitors for the treatment and prevention of cancer. To the contrary, emerging evidence indicates that COX-1 activity may contribute to carcinogenesis, so that maintaining pan-COX inhibition is likely to be more beneficial than selectively inhibiting COX-2. For example, the knocking out of the COX 1 gene also protects against the formation of intestinal and skin tumors in the Min mouse model. See Chulada et al. (Cancer Research; 60: 4705-08 2000) Thus, inhibiting both COX-1 and COX-2 is likely to be more beneficial than inhibiting COX-1 alone. However, in spite of this COX-1 knockout study, few if any researchers in the field have emphasized the potential role of COX-1 in carcinogenesis.

The growth of tumors typically is also associated with immune suppression. During the development of tumors, host monocytes and macrophages are triggered to produce high levels of prostaglandin E₂ (PGE₂) which has immunosuppressive effects. The immunosuppressive effects of PGE₂ include the inhibition of: T and B lymphocyte proliferation, lymphokine production, cytotoxicity of natural killer (NK) cells, effector functions of T-cells, B-cells, and macrophages, and generation of cytotoxic T lymphocytes and lymphokine-activated killer (LAK) cells.

Studies using prostaglandin synthesis inhibitors, like non-steroidal anti-inflammatory drugs (NSAID), have provided further evidence for the role of prostaglandins in mediating immunosuppression. Considerable evidence further suggests that NSAIDs may have an important role in chemoprevention. The use of NSAIDs has been shown effective in reducing or inhibiting tumor growth and bone metastasis.

For example, Hong et al., 2000, “Cyclooxygenase regulates human oropharyngeal carcinomas via the proinflammatory cytokine IL-6: a general role for inflammation?” FASEB J. 14:1499-1507 discloses that ketorolac, a pan-COX inhibitor and NSAID continuously and significantly decreased PGE₂ production and IL-6 and IL-8 levels in all OPC cell lines tested and reduced OPC growth in vivo but not in vitro. It is noted that ketorolac does play a role in decreasing inflammation. It is speculated that chronic inflammation may play a role in promoting the development of OPC and that perhaps the mechanism may apply to other epithelial tumor systems modulated by COX activity.

Falk et al., U.S. Pat. Nos. 6,136,793, 6,114,314, 6,103,704 and 6,218,373 disclose compositions suitable for topical application to the skin in the form of a gel or awn comprising nonsteroidal antinflammatory drugs (NSAIDs) and an effective amount of hyaluronic acid sufficient to transport the drug to a site of a disease or condition. NSAIDs disclosed included diclofenac, inomethacia, naproxen, and tromethamine salt of ketorolac, ibuprofen, piroxicam propionic acid derivatives, acetylsalicylic acid and finixin. These compositions are disclosed to be used to treat basal cell carcinoma, actinic keratoses lesions, fungal lesions, “liver” spots, squamous cell tumors, metastatic cancer of the breast to the skin, primary and metastatic melanoma in the skin, genital warts, cervical cancer, human papilloma virus of the cervix, psoriasis, corns of the feet and hair loss in pregnant women. It is thought that NSAID prevents the enzymatic production of prostaglandins, which block macrophage and Natural Killer (NK) cell functions in the local anti-tumor immune response. The hyaluronic acid is thought to act by transporting the NSAID with it until the space between the cells to the area of trauma. As a result, it enhances the activity of prostaglandin synthesis inhibition and reduces any side effects that are associated with the use of the NSAID.

Wechter et al., U.S. Pat. No. 5,955,504 has disclosed the use of R-NSAIDs primarily for the treatment and prevention of colorectal cancers. However, large amounts of R-NSAID need to be used.

Cavanaugh, U.S. Pat. No. Re No. 36,419 primarily discloses a method for using an NSAID and in particular, ketorolac for treatment of primary or recurring squamous cell carcinomas of the oral cavity or oropharynx by topical administration.

Seibert et al., U.S. patent application publication US 2001/0047024 A1 discloses a method of using cyclooxygenase-2 inhibitors in the treatment and prevention of neoplasia. In this publication, the term “cyclogenase-2 inhibitor” denotes a compound able to inhibit cyclogenoxygenase-2 without significant inhibition of cycloxygenase-1.

Fey et al., U.S. patent application publication US 2001/0011097 discloses a method of reducing or inhibiting mucositis by administering, for example, an NSAID in combination with an inflammatory cytokine inhibitor, mast cell inhibitor, an MMP inhibitor or an NO inhibitor.

### 3. OBJECTS OF THE INVENTION

It is an object of the invention to provide an effective means to prevent and treat cellular abnormalities of lung or bronchial pathway of a mammal that can be used alone or in conjunction with existing treatments.

It is a further object of the invention to provide a means for administering the NSAID to a mammal which results in high local concentration of the drug in the target tissue with minimal systemic (blood) concentration of said NSAID.

It is a further object of the invention to provide a means for administering the NSAID to a mammal that results in minimal exposure to the gastric epithelium so as to provide for reduced incidence of gastrointestinal toxicity including gastrointestinal hemorrhage.

### 4. SUMMARY OF THE INVENTION

The invention is directed to methods of using a non-steroidal anti-inflammatory drug (NSAID), to treat or prevent a cellular abnormality of the lung or bronchial pathway of a mammal comprising administering to said bronchial pathway in need thereof an amount of a non-steroidal anti-inflammatory drug (NSAID), alone or as an adjunct to chemotherapy, surgery and/or radiation therapy effective to treat said cellular abnormality. In a specific embodiment, the cellular abnormality is a squamous cell carcinoma as well as bronchial intraepithelial neoplasia, a precancerous condition. The invention is also directed to compositions comprising said NSAID wherein said composition is essentially free of hyaluronic acid. The invention is also directed to preventing metastases and/or secondary unrelated tumors comprising treating a mammal where a primary tumor has been removed with an NSAID or composition comprising said NSAID. As will be described in further detail below, the NSAID is preferably a COX-1 selective cyclooxygenase inhibitor, a non-selective cyclooxygenase inhibitor, or a partially selective cyclooxygenase-2 (COX-2) inhibitor. The invention is further directed to the use of said NSAIDs for use in the manufacture of a medicament for prevention or treatment of said cellular abnormality or for preventing said metastases or secondary cancers.

The systemic administration of high doses of NSAIDs, including the new generation of highly selective COX-2 inhibitors (like celecoxib), may cause adverse events such as ulcer complications, atria; fibrillation, and cardiac arrhythmia (Scrip # 2610 p12 (Feb. 21, 2001). Therefore, in order to minimize gastric as well as systemic exposure and maximize delivery to the site of action, the concept of using an NSAID-containing topical application was developed.

Local or topical delivery of NSAIDs is likely to achieve high local concentrations of NSAIDs in the target tissue. In a specific embodiment, the amount of NSAID administered topically is sufficiently high to access COX-independent pathways of antitumor effects. Other advantages of topical delivery of COX-1 selective cyclooxygenase inhibitors, nonselective cyclooxygenase inhibitors, and partially selective cyclooxygenase-2 (COX-2) inhibitors include reduced GI and systemic toxicity and improved therapeutic index. The concentration of the drug is high in the target tissue and the concentration in the bloodstream and the GI tract is minimized. Sufficient uptake to the mucosal epithelium is achieved without the use of hyaluronic acid or other carrier.

In addition, with topical administration, a much higher concentration of NSAID in the diseased tissue is obtained than when the NSAID is systemically administered. The levels of NSAID in the diseased tissue obtained through topical or local administration of NSAID enables accessing both COX-dependent and COX-independent antitumor effects. Furthermore, levels of NSAID sufficient to access certain COX-independent pathways would be impossible to achieve through systemic administration of the NSAID.

As defined herein, “essentially free of hyaluronic acid” means that the NSAID or composition comprising said NSAID does not contain any amounts of hyaluronic acid significant to affect the transport of the NSAID, preferably less than about 0.1% hyaluronic acid, and most preferably, those comprising less than about 0.01% hyaluronic acid.

“Treat” and “treatment”, as used herein, mean to attempt to slow the progress of or to reverse the symptoms of the condition being addressed.

### 5. DETAILED DESCRIPTION OF THE INVENTION

The method of the present invention is directed to using NSAIDs to treat and prevent cellular abnormalities of the lung or bronchial pathway of a mammal. In a specific embodiment, the mammal is a human patient.

5.1. NSAIDs

The NSAIDs used in the method of the present invention may be a COX-1 selective cyclooxygenase inhibitor, a nonselective cyclooxygenase inhibitor, or a partially selective cyclooxygenase-2 inhibitor. As defined herein, a “nonselective cyclooxygenase inhibitor” has a selectivity ratio of cyclooxygenase-2 inhibition over cyclooxygenase-1 inhibition of between about 0.1-15. Alternatively, the NSAIDs may be partially selective cyclooxygenase-2 (COX-2) inhibitors. As defined herein, a “partially selective cyclooxygenase-2 inhibitor” has a selectivity ratio of cyclooxygenase-2 inhibition over cyclooxygenase-1 inhibition of between 15-50. As defined herein, a “COX-1 selective cycloxygenase inhibitor” has a selectivity ratio of cyclooxygenase-2 inhibition over cyclooxygenase-1 inhibition of less than 0.1. The NSAIDs may include but are not limited to flurbiprofen, ketoprofen, fenoprofen, carprofen, diflunisal, piroxicam and sulindac, aspirin, ampyrone, ibuprofen, indomethacin, ketorolac, naprosen, niflumic acid, oxaprosin, suprofen, tenoxicam, tamoxifen, ticlopidine, tenidap, tolmetin, diclofenac, etodolac, flufenamate, meclofenamate, mefenamic acid, meloxicam, nabumetone, nimesulide, resveratrol, 6-MNA, zomepirac, and tomoxiprol, and mixtures thereof.

In a preferred embodiment, the NSAID is ketorolac. “Ketorolac”, as used herein, is (.+−.)5(benzoyl)-2,3-dihydro-1H-pyrrolizine-1carboxylic acid, and the pharmaceutically acceptable non-toxic esters and salts thereof, as disclosed in U.S. Pat. No. 4,089,969 issued to Muchowski & Kluge on May 16, 1978. The (−)-S enantiomer of ketorolac is preferred.

Pharmaceutically acceptable esters of ketorolac include but are not limited to, alkyl esters derived from hydrocarbons of branched or straight chain having one to about 12 carbon atoms. Examples of such esters are methyl, ethyl, propyl, isopropyl, butyl, t-butyl, isoamyl, pentyl, isopentyl, hexyl, octyl, nonyl, isodecyl, 6-methyldecyl and dodecyl esters.

Pharmaceutically acceptable salts of ketorolac include salts derived from either inorganic or organic bases. Salts derived from inorganic bases include sodium potassium, lithium ammonium, calcium, magnesium, ferrous, zinc, copper, manganese, aluminum, ferric, manganic salts and the like. Particularly preferred are the ammonium, potassium, sodium, and lithium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, tromethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic non-toxic bases are isopropylamine, diethylamine, ethanolamine, piperidine, tromethamine, dicyclohexylamine, choline and caffeine.

The preferred ketorolac salt, which is soluble in the composition of the subject invention in which it is incorporated, for use in the compositions and methods of the present invention is the racemic mixture of (+)R and (−)-S enantiomer of ketorolac tromethamine, and most preferred is its (−)-S enantiomer, (−)-5(benzoyl)-2,3-dihydro-1H-pyrrolizine-1-carboxylic acid, 2-amino-2-(hydroxymethyl)-1,3-propanediol.

5.2 Compositions

One aspect of the present invention is compositions comprising a safe and effective amount, preferably from about 0.001% to about 15%, 0.003% to about 10%, more preferably from about 0.005% to about 1%, more preferably still from about 0.01% to about 0.5%, even more preferably from about 0.1% to about 0.5%, still more preferably from about 0.05% to about 0.2% ketorolac, and a pharmaceutically acceptable carrier or excipients. These include flavoring agents, diluents, emulsifiers, dispersing aids or binders, buffers, diluents and other suitable additives such as, but not limited to, penetration enhancers, as well as a breathable liquid carrier selected from the group consisting of fluorocarbon liquids, saline, silicone liquids, vegetable oils and combinations thereof. As used herein, the phrase “breathable liquids” refers to liquids that have the ability to deliver oxygen into, and to remove carbon dioxide from, the pulmonary system (i.e., the lungs) of patients. The carrier may also be an aqueous pH buffered solution, for example, buffers such as phosphate, citrate and other organic acids. Other examples o physiologically acceptable carriers include but are not limited to antioxidants including acorbic acid, low molecular weight polypeptide, proteins, such as serum albumin, gelatin, or immunoglobulins; hydorphilic polymers such as polyvinylpyrrolidone, amino acids such as glycine, glutamine, asparagine, arginine or lysine; monosaccharides, disaccharides and other carbohydrates including glucose, mannose or dextrins; chelating agents such as EDTA; sugar alcohols such as mannitol or sorbitol; salt forming counterions such as sodium; and/or nonionic surfactants such as TWEEN™, polyethylene glycol (PEG) and PLURONICS™. Conventional methods are used in preparing the compositions.

In a preferred embodiment, the compositions and NSAIDs may be administered to the bronchial pathway via pulmonary delivery devices or means known in the art as described, for example, in U.S. Pat. No. 5,707,352 or the AIRs™ system marketed by Alkermes which involves the use of dry microspheres made of polylactic acid material or using the device described in U.S. Pat. No. 6,348,209 (inhalation device). The compositions may be delivered in the form of an aerosol spray of liquid or liquid. These include but are not limited to catheters, bronchial tubes, and liquid lavage/ventilation. The NSAIDs or compositions of the present invention may also be in the form or

powders or emulsions dissolved in liquid carriers or aerosolized for delivery to the bronchial tissues. Additionally, the NSAIDs may be administered via a device where the particle size of the formulations and compositions comprising said NSAID are modulated so that the particles would go to varying locations dependent on its size. For example large particles would go to the deep lung and smaller particles would go to the bronchial tubes. Generally, the optimal size range for drug delivery into the tracheobronchial and pulmonary reigions is about 1-5 microns. Particles having an aerodiameter greater than 5 microns are typically deposited in the nasopharyngeal region.

NSAIDs of the present invention may additionally be combined with chemotherapeutic agents or further antimicrobial (antiviral, antibacterial or antifungal) or immunomodulatory compounds to provide a combination therapy. Combination therapy is intended to include any chemically compatible combination of an NSAID of the present invention with other compounds of the present invention or other compounds outside of the present invention, as long as the combination does not eliminate the activity of the NSAID of the present invention. For example, one or more NSAIDs of the present invention may be combined with vasoconstrictors, vasodilators, bronchoconstrictors, bronchodilators, anti-cancer agents, steroids, antimicrobial agents, chemotactic agents or chemotherapeutic agents (e.g., adriamycin, toxins, antibody-linked nuclides, etc.). Combination therapy can be sequential, that is the treatment with one agent first and then the second agent, or it can be treatment with both agents at the same time. The NSAIDs and the second agent may be combined into one composition. The sequential therapy can be within a reasonable time after the completion of the first therapy before beginning the second therapy.

The compositions of the present invention should be administered at least one per day but may be administered up to about four times daily or even more frequently. The specific treatment regimen will be dependent on the nature of the condition being treated. The treatment may be for as short as three months and could continue for up to five years, but preferably about six months.

The compositions of the present invention may be held in the pulmonary for a period of from 15 seconds to about 48 hours. The compositions of the present invention should have favorable tissue residence times. The time the drug persists in the tissue is the residence time. A favorable residence time is a time that allows for convenient dosing and maintains sufficient tissue drug concentration to inhibit the COX enzymes and/or reduce abnormal cell growth.

5.3 Uses

The NSAIDs and compositions of the present invention may be used to treat cellular abnormalities of the lung or bronchial pathway of a mammal. This may include cancers as well as precancerous lesions such as hyperplasia, metaplasia, or most particularly, dysplasia (for review of such abnormal growth conditions, see Robbins and Angell, 1976, Basic Pathology, 2d Ed., W. B. Saunders Co., Philadelphia, pp. 68-79.) Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. In particular, the compositions and NSAIDs may be used to prevent the growth of precancerous squamous lung cell carcinoma cells.

The NSAIDs and compositions of the present invention may also be used to treat lung cancer, or bronchial carcinoma, particularly tumors arising from the major airways (bronchi) and pulmonary parenchyma (bronchioles, alveoli, and supporting tissue), as opposed to those metastasizing from other sites. The lung cancers that may be treated include but are not limited to, squamous cell carcinoma (SCC), adenocarcinoma (AC), large cell anaplastic carcinoma (LCAC), and small cell anaplastic carcinoma (SCAC). The NSAIDs may be used to prevent metastases or the occurrence of nonrelated secondary tumors in patients where the primary tumor has been removed.

The specific embodiments herein disclosed are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Various references are cited herein, the disclosures of which are incorporated by reference in their entireties.

What is claimed is:

1. A method of preventing or treating a cellular abnormality of the lung or bronchial pathway of a mammal comprising administering to the bronchial pathway of said mammal in need thereof an amount of an NSAID effective, alone or as an adjunct to chemotherapy, surgery and/or radiation therapy to prevent or treat said cellular abnormality, wherein said NSAIDs are selected from the group consisting of COX-1 selective cyclooxygenase inhibitors, nonselective cyclooxygenase inhibitors, and partially selective cyclooxygenase-2 (COX-2) inhibitors.

2. The method according to claim 1, wherein the cellular abnormality is a precancerous lesion (including intraepithelial neoplasia and dysplasia) of the lung or bronchial pathway.

3. The method according to claim 1, wherein the cellular abnormality is carcinoma in situ of the lung or bronchial pathway.

4. The method according to claim 1, wherein the cellular abnormality is lung cancer.

5. The method according to claim 3, wherein said lung cancer is selected from the group consisting of squamous cell carcinoma (SCC), adenocarcinoma (AC), large cell anaplastic carcinoma (LCAC), and small cell anaplastic carcinoma (SCAC).

6. The method according to claim 1, wherein an aerosolized spray is applied to said bronchial pathway.

7. The method according to claim 5, wherein the aerosolized spray is applied to said bronchial pathway for a period of from about 15 seconds to about 10 minutes.

8. The method according to claim 1 wherein the NSAID is selected from the group consisting of flurbiprofen, ketoprofen, fenoprofen, carprofen, diflunisal, piroxicam and sulindac, aspirin, ampyrone, ibuprofen, indomethacin, ketorolac, naprosen, niflumic acid, oxaprosin, suprofen, tenoxicam, tamoxifen, ticlopidine, tenidap, tolmetin, diclofenac, etodolac, flufenamate, meclofenamate, mefenamic acid, meloxicam, nabumetone, nimesulide, resveratrol, 6-MNA, zomepirac, and tomoxiprol, and mixtures thereof.

9. The method according to claim 1, wherein the NSAID is ketorolac.

10. The method according to claim 8, wherein the ketorolac is ketorolac tromethamine.

11. The method according to claim 8, wherein the ketorolac is an S-enantiomer of ketorolac tromethamine.

12. The method according to claim 1, wherein the mammal is a human.

13. The method according to claim 1, wherein the cellular abnormality is a precancerous squamous lung cell carcinoma condition.

14. The method according to claim 1 in which the NSAID is administered in an amount sufficiently high to access COX-independent pathways of antitumor effects.

15. A method of preventing or treating a cellular abnormality of the lung or bronchial pathway of a mammal comprising administering to the bronchial pathway of said mammal an amount of a composition comprising comprising about 0.001% to about 10%, by weight of an NSAID, essentially free of hyaluronic acid, alone or as an adjunct to surgery and/or radiation therapy in an amount effective to prevent or treat said cellular abnormality, wherein said NSAIDs are selected from the group consisting of COX-1 selective cyclooxygenase inhibitors, nonselective cyclooxygenase inhibitors, and partially selective cyclooxygenase-2 (COX-2) inhibitors.

16. A method for preventing metastases or unrelated secondary tumor in a patient having a primary tumor in the lung or bronchial pathway, wherein said primary tumor has been removed comprising administering to said patient an amount of an NSAID effective to prevent said metastases or unrelated secondary tumor, wherein said NSAIDs are selected from the group consisting of COX-1 selective cyclooxygenase inhibitors, nonselective cyclooxygenase inhibitors, and partially selective cyclooxygenase-2 (COX-2) inhibitors.

17. A composition essentially free of hyaluronic acid comprising an NSAID and a breathable liquid carrier selected from the group consisting of fluorocarbon liquids, saline, silicone liquids, vegetable oils and combinations thereof, wherein said NSAIDs are selected from the group consisting of COX-1 selective cyclooxygenase inhibitors, nonselective cyclooxygenase inhibitors, and partially selective cyclooxygenase-2 (COX-2) inhibitors.

18. The composition according to claim 17, wherein said composition further comprises an agent selected from the group consisting of from the group consisting of vasoconstrictors, vasodilators, bronchoconstrictors, immunomodulator, anti-cancer agents, steroids, antimicrobial agents, chemotactic agents, chemotherapeutic agents, and combinations thereof.

19. The composition according to claim 17, which further comprises cytokines or chemokines.
